(19) United States
(12) Patent Application Publication
(10) Pub. No.: US 2009/0005332 A1
(43) Pub. Date: Jan. 1, 2009

(54) Compositions and Methods for Modulating Gene Expression Using Self-Protected Oligonucleotides

(76) Inventors: Todd M. Hauser, Sammamish, WA (US); Aaron Loomis, Prairie Du Sac, WI (US)

Correspondence Address: SEED INTELLECTUAL PROPERTY LAW GROUP PLLC, 701 FIFTH AVE, SUITE 5400, SEATTLE, WA, 98104, US

(21) Appl. No.: 11/813,190

(22) PCT Filed: Dec. 30, 2005
(86) PCT No.: PCT/US05/47610

§ 371 Date: May 16, 2008

Related U.S. Application Data
(60) Provisional application No. 60/640,584, filed on Dec. 30, 2004.

(51) Int. Cl. A61K 31/711; C07H 21/02; C07H 21/04; C40B 40/08; A61P 35/00; A61P 31/00; C12Q 1/68; C12N 15/63; A61K 31/7105

(52) U.S. Cl. 514/44; 536/23.1; 506/17; 435/320.1; 435/6

(57) ABSTRACT

The present invention is directed to novel nucleic acid molecules which include a region complementary to a target gene and one or more self-complementary regions, and the use of such nucleic acid molecules and compositions comprising the same to modulate gene expression and treat a variety of diseases and infections.

### BACKGROUND OF THE INVENTION

1. Field of the Invention

The present invention relates generally to self-protected polynucleotides, and methods of using the same to modulate gene expression and treat disease.

2. Description of the Related Art

The phenomenon of gene silencing, or inhibiting the expression of a gene, holds significant promise for therapeutic and diagnostic purposes, as well as for the study of gene function itself. Examples of this phenomenon include antisense technology and posttranscriptional gene silencing (PTGS).

Antisense strategies for gene silencing have attracted much attention in recent years. The underlying concept is simple yet, in principle, effective: antisense nucleic acids (NA) base pair with a target RNA resulting in inactivation. Target RNA recognition by antisense RNA or DNA can be considered a hybridization reaction. Since the target is bound through sequence complementarity, this implies that an appropriate choice of antisense NA should ensure high specificity. Inactivation of the targeted RNA can occur via different pathways, dependent on the nature of the antisense NA (either modified or unmodified DNA or RNA) and on the properties of the biological system in which inhibition is to occur.

However, many problems persist in the development of effective antisense and PTGS technologies. For example, DNA antisense oligonucleotides exhibit only short-term effectiveness and are usually toxic at the doses required; similarly, the use of antisense RNAs has also proved ineffective due to stability problems. Various methods have been employed in attempts to improve antisense stability by reducing nuclease sensitivity. These include modifying the normal phosphodiester backbone, e.g., using phosphorothioates or methyl phosphonates, incorporating 2′-OMe-nucleotides, using peptide nucleic acids (PNAs) and using 3′-terminal caps, such as 3′-aminopropyl modifications or 3′-3′ terminal linkages. However, these methods can be expensive and require additional steps. In addition, the use of non-naturally occurring nucleotides and modifications precludes the ability to express the antisense sequences in vivo, thereby requiring them to be synthesized and administered afterwards.

Consequently, there remains a need for effective and sustained methods and compositions for the targeted, directed inhibition of gene function in vitro and in vivo, particularly in cells of higher vertebrates, including improved antisense RNAs having increased stability.

### BRIEF SUMMARY OF THE INVENTION

The present invention provides novel compositions and methods, which include self-protected oligonucleotides useful in regulating gene expression.

In one embodiment, the present invention includes an isolated self-protected polynucleotide comprising a region having a sequence complementary to a target mRNA sequence and one or more self-complementary regions. In particular embodiments, the oligonucleotide comprises two or more self-complementary regions. The self-complementary regions may be located at the 5′ end, the 3′ end, or both ends of the polynucleotide.

In certain embodiments, self-protected polynucleotides of the present invention comprise RNA, DNA, or peptide nucleic acids.

In additional embodiments, a self-protected polynucleotide further comprises a second sequence that is non-complementary or semi-complementary to a target mRNA sequence and non-complementary to a self-complementary region, and said second sequence is located between the self-complementary region and the sequence complementary to a target mRNA sequence.

In particular embodiments, a self-complementary region comprises a stem-loop structure.

In related embodiments, a self-complementary region is not complementary to the sequence complementary to a target mRNA sequence.

In further related embodiments, wherein the polynucleotide comprises two self-complementary regions, the two self-complementary regions do not complement each other.

In particular embodiments, the sequence complementary to a target mRNA sequence comprises at least 17 nucleotides, or 17 to 30 nucleotides.

In other embodiments, the self-complementary region comprises at least 5 nucleotides, at least 12 nucleotides, at least 24 nucleotides, or 12 to 48 nucleotides.

In further embodiments, a loop region of a stem-loop structure comprises at least 1 nucleotide. In other embodiments, the loop region comprises at least 2, at least 3, at least 4, at least 5, or at least 6 nucleotides.

In another embodiment, the present invention includes an array comprising a plurality of self-protected polynucleotides of the present invention.

In a further embodiment, the present invention includes an expression vector capable of expressing a self-protected polynucleotide of the present invention. In various embodiments, the expression vector is a constitutive or an inducible vector.

The present invention further includes a composition comprising a physiologically acceptable carrier and a self-protected polynucleotide of the present invention.

In other embodiments, the present invention provides a method for reducing the expression of a gene, comprising introducing self-protected oligonucleotides of the present invention into a cell. In various embodiments, the cell is plant, animal, protozoan, viral, bacterial, or fungal. In one embodiment, the cell is mammalian.

In some embodiments, the polynucleotide is introduced directly into the cell, while in other embodiments, the polynucleotide is introduced extracellularly by a means sufficient to deliver the isolated polynucleotide into the cell.

In another embodiment, the present invention includes a method for treating a disease, comprising introducing a self-protected polynucleotide of the present invention into a cell, wherein overexpression of the targeted gene is associated with the disease. In one embodiment, the disease is a cancer.

The present invention further provides a method of treating an infection in a patient, comprising introducing into the patient a self-protected polynucleotide of the present invention, wherein the isolated polynucleotide mediates entry, replication, integration, transmission, or maintenance of an infective agent.

In yet another related embodiment, the present invention provides a method for identifying a function of a gene, comprising introducing into a cell a self-protected oligonucleotides of the present invention, wherein the polynucleotide inhibits expression of the gene, and determining the effect of the introduction of the polynucleotide on a characteristic of the cell, thereby determining the function of the targeted gene. In one embodiment, the method is performed using high throughput screening.

In a further embodiment, the present invention provides a method of designing a polynucleotide sequence comprising one or more self-complementary regions for the regulation of expression of a target gene, comprising: (a) selecting a first sequence 17 to 30 nucleotides in length and complementary to a target gene; (b) selecting one or more additional sequences 12 to 48 nucleotides in length, which comprises self-complementary regions and which are non-complementary to the first sequence; and (c) selecting one or more further additional sequences 2 to 12 nucleotides in length, which are non-complementary or self-complementary to the target gene and which are non-complementary to the additional sequences selected in step (b).

In another embodiment, a self-protected polynucleotide of the present invention exhibits an increased half-life in vivo, as compared to the same polynucleotide lacking the one or more self-complementary regions.

In a further related embodiment, the present invention provides a method for treating a disease (e.g., a disease associated with a mutated mRNA or gene), comprising introducing a self-protected polynucleotide into a cell, wherein the targeted mRNA comprises one or more mutations as compared to a corresponding wild-type mRNA. In one specific embodiment, the disease is cystic fibrosis.

Similarly, in a related embodiment, the invention includes a method of modulating the expressing of a mutated mRNA in a cell, comprising introducing a self-protected polynucleotide into a cell, wherein said target RNA sequence comprises a region of said mutated mRNA. In one specific embodiment, the mutated mRNA is associated with cystic fibrosis. In a specific embodiment, the mutated mRNA is an mRNA expressed from a gene encoding a mutant Cystic Fibrosis Transmembrane Conductance Regulator (CFTR) polypeptide. In another embodiment, the mutated mRNA is associated with a tumor. In another embodiment, the mutated mRNA is an mRNA expressed from a gene encoding a mutant p53 polypeptide.

### BRIEF DESCRIPTION OF THE SEVERAL VIEWS OF THE DRAWING(S)

FIG. 1 provides a diagram of an exemplary polynucleotide of the present invention. (A) indicates the region comprising sequence complementary to a target mRNA; (B) indicates a self-complementary region; (C) indicates a loop region of a stem-loop structure formed by the self-complementary region; and (D) indicates a gap region between the region comprising sequence complementary to a target mRNA and a self-complementary region.

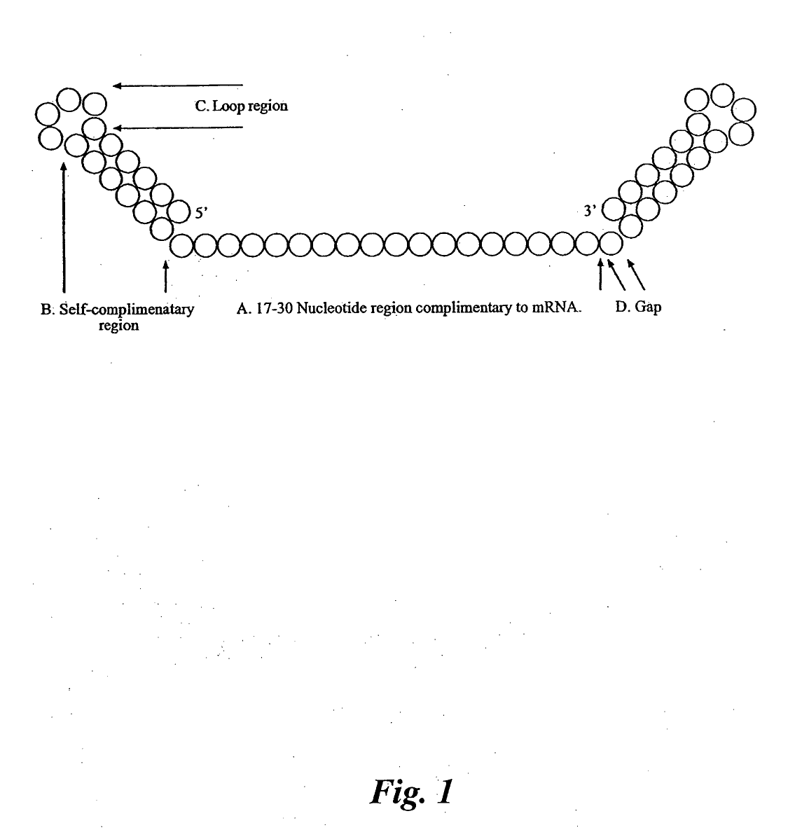

### DETAILED DESCRIPTION OF THE INVENTION

The present invention provides novel compositions and methods for inhibiting the expression of a target gene in prokaryotes and eukaryotes in vivo and in vitro.

The invention is based, in part, upon the surprising discovery that antisense oligonucleotides further comprising one or more self-complementary regions capable of forming a stem-loop structure are more stable and markedly more effective in inhibiting expression of a target gene. In specific embodiments, the present invention provides a composition that is effective to inhibit the expression of the targeted gene in vitro or in vivo. Given their increased effectiveness, the compositions of the present invention may be delivered to a cell or subject at lower concentrations with an accompanying reduced toxicity.

### A. Self-Protected Polynucleotides

In accordance with the present invention, self-protected polynucleotides, which comprise a nucleotide sequence with complementarity to an mRNA expressed from a target gene, as well as one or more self-complementary regions, are used to regulate gene expression. As used herein, a self-protected polynucleotide means an isolated polynucleotide comprising a single-stranded region complementary to a region of a target mRNA or gene sequence, and one or more self-complementary regions located at one or both the 5′ or 3′ ends of the polynucleotide, which are capable of forming a double-stranded region, such as a stem-loop structure. Self-protected polynucleotides are also referred to herein as self-protected oligonucleotides. Self-protected polynucleotides of the present invention offer surprising advantages over polynucleotide inhibitors of the prior art, including antisense RNA and RNA interference molecules, including increased stability and increased effectiveness.

In certain embodiments, self-protected polynucleotides comprise two or more regions of sequence complementary to a target gene. In particular embodiments, these regions are complementary to the same target genes or mRNAs, while in other embodiments, they are complementary to two or more different target genes or mRNAs. Accordingly, the present invention includes self-complementary polynucleotides comprising a series of sequences complementary to one or more target mRNAs or genes. In particular embodiments, these sequences are separated by regions of sequence that are non-complementary or semi-complementary to a target mRNA sequence and non-complementary to a self-complementary region. In other embodiments of self-protected polynucleotides comprising multiple sequence complementary to target genes or mRNAs, the self-protected polynucleotide comprises a self-complementary region at the 5′, 3′, or both ends of one or more regions of sequence complementary to a target gene. In a particular embodiment, a self-protected polynucleotide comprises two or more regions of sequence complementary to one or more target genes, with self-complementary regions located at the 5′ and 3′ end of each region complementary to a target gene.

As used herein, the term “self-complimentary” refers to a nucleotide sequence wherein a first region of the nucleotide sequence binds to a second region of the nucleotide sequence to form A-T(U) and G-C hybridization pairs. The two regions of the nucleotide sequence that bind to each other may be contiguous or may be separated by other nucleotides. The term “non-complimentary” indicates that in a particular stretch of nucleotides, there are no nucleotides within that align with a target to form A-T(U) or G-C hybridizations. The term “semi-complimentary” indicates that in a stretch of nucleotides, there is at least one nucleotide pair that aligns with a target to form an A-T(U) or G-C hybridizations, but there is not a sufficient number of complementary nucleotide pairs to support binding within the stretch of nucleotides under physiological conditions.

The term isolated refers to a material that is at least partially free from components that normally accompany the material in the material's native state. Isolation connotes a degree of separation from an original source or surroundings. Isolated, as used herein, e.g., related to DNA, refers to a polynucleotide that is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man.

In various embodiments, a self-protected polynucleotide of the present invention comprises RNA, DNA, or peptide nucleic acids, or a combination of any or all of these types of molecules. In addition, a self-protected polynucleotide may comprise modified nucleic acids, or derivatives or analogs of nucleic acids.

Examples of nucleic acid modifications include, but are not limited to, biotin labeling, fluorescent labeling, amino modifiers introducing a primary amine into the polynucleotide, phosphate groups, deoxyuridine, halogenated nucleosides, phosphorothioates, 2′-OMe RNA analogs, chimeric RNA analogs, wobble groups, and deoxyinosine.

The term “analog” as used herein refers to a molecule, compound, or composition that retains the same structure and/or function (e.g., binding to a target) as a polynucleotide herein. Examples of analogs include peptidomimetics, peptide nucleic acids, and small and large organic or inorganic compounds.

The term “derivative” or “variant” as used herein refers to a polynucleotide that differs from a naturally occurring polynucleotide (e.g., target gene sequence) by one or more nucleic acid deletions, additions, substitutions or side-chain modifications. In certain embodiments, variants have at least 70%, at least 80% at least 90%, at least 95%, or at least 99% sequence identity to a region of a target gene sequence. Thus, for example, in certain embodiments, a self-protected oligonucleotide of the present invention comprises a region that is complementary to a variant of a target gene sequence.

In each case, self-protected polynucleotides of the present invention comprise a sequence region that is complementary, and more preferably, completely complementary to one or more regions of a target gene or polynucleotide sequence (or a variant thereof). In certain embodiments, selection of a sequence region complementary to a target gene (or mRNA) is based upon analysis of the chosen target sequence and determination of secondary structure, Tₘ, binding energy, and relative stability. Such sequences may be selected based upon their relative inability to form dimers, hairpins, or other secondary structures that would reduce or prohibit specific binding to the target mRNA in a host cell. Highly preferred target regions of the mRNA include those regions at or near the AUG translation initiation codon and those sequences that are substantially complementary to 5′ regions of the mRNA. These secondary structure analyses and target site selection considerations can be performed, for example, using v.4 of the OLIGO primer analysis software and/or the BLASTN 2.0.5 algorithm software (Altschul et al., Nucleic Acids Res. 1997, 25(17):3389-402).

In another embodiment, target sites are selected by scanning the target mRNA transcript sequence for the occurrence of AA dinucleotide sequences. Each AA dinucleotide sequence in combination with the 3′ adjacent approximately 19 nucleotides are potential siRNA target sites. In one embodiment, target sites are preferentially not located within the 5′ and 3′ untranslated regions (UTRs) or regions near the start codon (within approximately 75 bases), since proteins that bind regulatory regions may interfere with the binding of the polynucleotide. In addition, potential target sites may be compared to an appropriate genome database, such as BLASTN 2.0.5, available on the NCBI server at www.ncbi.nlm, and potential target sequences with significant homology to other coding sequences eliminated.

The target gene or mRNA may be of any species, including, for example, plant, animal (e.g. mammalian), protozoan, viral, bacterial or fungal.

As noted above, the target gene sequence and the complementary region of the self-protected polynucleotide may be complete complements of each other, or they may be less than completely complementary, as long as the strands hybridize to each other under physiological conditions.

Self-protected polynucleotides of the present invention comprise a region complementary to a target mRNA or gene, as well as one or more self-complementary regions. In addition, they may optionally comprise one or more gap regions located between the region complementary to a target mRNA or gene and a self-complementary region.

Typically, the region complementary to a target mRNA or gene is 17 to 30 nucleotides in length, including integer values within these ranges. This region may be at least 16 nucleotides in length, at least 17 nucleotides in length, at least 20 nucleotides in length, at least 24 nucleotides in length, between 16 and 24 nucleotides in length, or between 17 and 24 nucleotides in length, including any integer value within these ranges.

The self-complementary region is typically between 14 and 30 nucleotides in length, at least 14 nucleotides in length, at least 16 nucleotides in length, or at least 20 nucleotides in length, including any integer value within any of these ranges. In certain embodiments, self-complementary region is located at the 5′ or 3′ end of the polynucleotide. When the polynucleotide comprises two self-complementary regions, in certain embodiments, one is located at the 5′ end and one is located at the 3′ end.

In preferred embodiments, a self-complementary region is long enough to form a double-stranded structure. In one embodiment, a self-complementary region forms a stem-loop structure comprising a double-stranded region of self-complementary sequence and a loop of single-stranded sequence. Accordingly, in one embodiment, the primary sequence of a self-complementary region comprises two stretches of sequence complementary to each other separated by additional sequence that is not complementary or is semi-complementary. While less optimal, the additional sequence can be complementary in certain embodiments. The additional sequence forms the loop of the stem-loop structure and, therefore, must be long enough to facilitate the folding necessary to allow the two complementary stretches to bind each other. In particular embodiments, the loop sequence comprises at least 3, at least 4, at least 5 or at least 6 bases. In one embodiment, the loop sequence comprises 4 bases. The two stretches of sequence complementary to each other (within the self-complementary region; i.e., the stem regions) are of sufficient length to specifically hybridize to each other under physiological conditions. In certain embodiments, each stretch comprises 4 to 12 nucleotides; in other embodiments, each stretch comprises at least 4, at least 5, at least 6, at least 8, or at least 10 nucleotides, or any integer value within these ranges. In a particular embodiment, a self-complementary region comprises two stretches of at least 4 complementary nucleotides separated by a loop sequence of at least 4 nucleotides. Furthermore, when a self-protected polynucleotide comprises two or more self-complementary regions, in preferred embodiments, the two regions are not complementary to each other. Additionally, in preferred embodiments, a self-complementary region is not complementary to the region of the self-protected polynucleotide that is complementary to the target mRNA or gene.

In certain embodiments, the optional gap region comprises at least 1, at least 2, at least 3, or at least 4 nucleotides in length, or between 1 and 6 nucleotides in length, including any integer value falling within these ranges. In various embodiments, the gap region is not complementary to the target mRNA or gene, or it is semi-complimentary to the target mRNA or gene. In one embodiment, the gap region is not complimentary to a stem region of a self-complimentary region.

In particular embodiments, self-complementary regions possess thermodynamic parameters appropriate for binding of self-complementary regions, e.g., to form a stem-loop structure.

In one embodiment, self-complimentary regions are dynamically calculated by use of RNA via free-energy analysis and then compared to the energy contained within the remaining “non self-complimentary region” or loop region to ensure that the energy composition is adequate to form a desired structure, e.g., a stem-loop structure. In general, different nucleotide sequences of the mRNA targeting region are considered in determining the compositions of the stem-loops structures to ensure the formation of such. The free-energy analysis formula may again be altered to account for the type of nucleotide or pH of the environment in which it is used. Many different secondary structure prediction programs are available in the art, and each may be used according to the invention. Thermodynamic parameters for RNA and DNA bases are also publicly available in combination with target sequence selection algorithms, of which several are available in the art.

In one embodiment, the self-protected polynucleotide comprises or consists of (a) a sequence comprising 17 to 30 nucleotides in length (including any integer value in-between), which is complementary to and capable of hybridizing under physiological conditions to at least a portion of an mRNA molecule, flanked by (b) two gap regions comprising 1 to 4 nucleotide in length, and (c) two self-complementary sequences comprising 16 to 24 nucleotides in length (including any integer value in-between). In one embodiment, each self-complementary sequence is capable of forming a stem-loop structure, one of which is located at the 5′ end and one of which is located at the 3′ end of the self-protected polynucleotide.

In certain embodiments, the self-complementary region functions to inhibit or reduce degradation of the self-protected oligonucleotide under physiological conditions, such as the conditions within a cell. Without wishing to be bound to a particular theory, it is believed that the structure adopted by a self-complementary region makes the polynucleotide more resistant to nuclease degradation than those lacking a self-complementary region. In addition, the presence of the structure adopted by the self-complementary regions is believed to facilitate cellular uptake and reduce undesired side effects. Accordingly, in various embodiments, a self-protected polynucleotide has an increased in vivo half-life as compared to the same polynucleotide lacking self-complementary regions, as described herein. The half-life may be increased by at least 2-fold, at least 3-fold, at least 4-fold, at least 5-fold, or at least 10-fold, in various embodiments.

In preferred embodiments, self-protected polynucleotides of the present invention bind to and reduce expression of a target mRNA. A target gene may be a known gene target, or, alternatively, a target gene may be not known, i.e., a random sequence may be used. In certain embodiments, target mRNA levels are reduced at least 10%, at least 20%, at least 30%, at least 40%, at least 50%, at least 60%, at least 70%, at least 75%, at least 80%, at least 90%, or at least 95%.

In one embodiment of the invention, the level of inhibition of target gene expression (i.e., mRNA expression) is at least 90%, at least 95%, at least 98%, at least 99% or is almost 100%, and hence the cell or organism will in effect have the phenotype equivalent to a so-called “knock out” of a gene. However, in some embodiments, it may be preferred to achieve only partial inhibition so that the phenotype is equivalent to a so-called “knockdown” of the gene. This method of knocking down gene expression can be used therapeutically or for research (e.g., to generate models of disease states, to examine the function of a gene, to assess whether an agent acts on a gene, to validate targets for drug discovery).

The invention further provides arrays of self-protected polynucleotides of the invention, including microarrays. Microarrays are miniaturized devices typically with dimensions in the micrometer to millimeter range for performing chemical and biochemical reactions and are particularly suited for embodiments of the invention. Arrays may be constructed via microelectronic and/or microfabrication using essentially any and all techniques known and available in the semiconductor industry and/or in the biochemistry industry, provided only that such techniques are amenable to and compatible with the deposition and/or screening of polynucleotide sequences.

Microarrays of the invention are particularly desirable for high throughput analysis of multiple self-protected polynucleotides. A microarray typically is constructed with discrete region or spots that comprise self-protected polynucleotides of the present invention, each spot comprising one or more self-protected polynucleotide, preferably at positionally addressable locations on the array surface. Arrays of the invention may be prepared by any method available in the art. For example, the light-directed chemical synthesis process developed by Affymetrix (see, U.S. Pat. Nos. 5,445,934 and 5,856,174) may be used to synthesize biomolecules on chip surfaces by combining solid-phase photochemical synthesis with photolithographic fabrication techniques. The chemical deposition approach developed by Incyte Pharmaceutical uses pre-synthesized cDNA probes for directed deposition onto chip surfaces (see, e.g., U.S. Pat. No. 5,874,554).

In certain embodiments, a self-protected polynucleotide of the present invention is synthesized using techniques widely available in the art. In other embodiments, it is expressed in vitro or in vivo using appropriate and widely known techniques. Accordingly, in certain embodiments, the present invention includes in vitro and in vivo expression vectors comprising the sequence of a self-protected polynucleotide of the present invention. Methods well known to those skilled in the art may be used to construct expression vectors containing sequences encoding a self-protected polynucleotide, as well as appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.

Expression vectors typically include regulatory sequences, which regulate expression of the self-protected polynucleotide. Regulatory sequences present in an expression vector include those non-translated regions of the vector, e.g., enhancers, promoters, 5′ and 3′ untranslated regions, which interact with host cellular proteins to carry out transcription and translation. Such elements may vary in their strength and specificity. Depending on the vector system and cell utilized, any number of suitable transcription and translation elements, including constitutive and inducible promoters, may be used. In addition, tissue- or -cell specific promoters may also be used.

For expression in mammalian cells, promoters from mammalian genes or from mammalian viruses are generally preferred. In addition, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

In certain embodiments, the invention provides for the conditional expression of a self-protected polynucleotide. A variety of conditional expression systems are known and available in the art for use in both cells and animals, and the invention contemplates the use of any such conditional expression system to regulate the expression or activity of a self-protected polynucleotide. In one embodiment of the invention, for example, inducible expression is achieved using the REV-TET system. Components of this system and methods of using the system to control the expression of a gene are well documented in the literature, and vectors expressing the tetracycline-controlled transactivator (tTA) or the reverse tTA (rtTA) are commercially available (e.g., pTet-Off, pTet-On and ptTA-2/3/4 vectors, Clontech, Palo Alto, Calif.). Such systems are described, for example, in U.S. Pat. No. 5,650,298, No. 6271348, No. 5922927, and related patents, which are incorporated by reference in their entirety.

In one particular embodiment, self-protected polynucleotides are expressed using a vector system comprising a pSUPER vector backbone and additional sequences corresponding to the self-protected polynucleotide to be expressed. The pSUPER vectors system has been shown useful in expressing siRNA reagents and downregulating gene expression (Brummelkamp, T. T. et al., Science 296:550 (2002) and Brummelkamp, T. R. et al., Cancer Cell, published online Aug. 22, 2002). PSUPER vectors are commercially available from OligoEngine, Seattle, Wash.

### B. Methods of Regulating Gene Expression

Self-protected polynucleotides of the invention may be used for a variety of purposes, all generally related to their ability to inhibit or reduce expression of a target gene. Accordingly, the invention provides methods of reducing expression of one or more target genes comprising introducing a self-protected polynucleotide of the invention into a cell that contains a target gene or a homolog, variant or ortholog thereof. In addition, self-protected polynucleotides may be used to reduce expression indirectly. For example, a self-protected polynucleotide may be used to reduce expression of a transactivator that drives expression of a second gene, thereby reducing expression of the second gene. Similarly, a self-protected polynucleotide may be used to increase expression indirectly. For example, a self-protected polynucleotide may be used to reduce expression of a transcriptional repressor that inhibits expression of a second gene, thereby increasing expression of the second gene.

In various embodiments, a target gene is a gene derived from the cell into which a self-protected polynucleotide is to be introduced, an endogenous gene, an exogenous gene, a transgene, or a gene of a pathogen that is present in the cell after transfection thereof. Depending on the particular target gene and the amount of the self-protected polynucleotide delivered into the cell, the method of this invention may cause partial or complete inhibition of the expression of the target gene. The cell containing the target gene may be derived from or contained in any organism (e.g., plant, animal, protozoan, virus, bacterium, or fungus).

Inhibition of the expression of the target gene can be verified by means including, but not limited to, observing or detecting an absence or observable decrease in the level of protein encoded by a target gene, and/or mRNA product from a target gene, and/or a phenotype associated with expression of the gene, using techniques known to a person skilled in the field of the present invention.

Examples of cell characteristics that may be examined to determine the effect caused by introduction of a self-protected polynucleotide of the invention include, cell growth, apoptosis, cell cycle characteristics, cellular differentiation, and morphology.

A self-protected polynucleotide may be directly introduced to the cell (i.e., intracellularly), or introduced extracellularly into a cavity, interstitial space, into the circulation of an organism, introduced orally, by bathing an organism in a solution containing the self-protected polynucleotide, or by some other means sufficient to deliver the self-protected polynucleotide into the cell.

In addition, a vector engineered to express a self-protected polynucleotide may be introduced into a cell, wherein the vector expresses the self-protected polynucleotide, thereby introducing it into the cell. Methods of transferring an expression vector into a cell are widely known and available in the art, including, e.g., transfection, lipofection, scrape-loading, electroporation, microinjection, infection, gene gun, and retrotransposition. Generally, a suitable method of introducing a vector into a cell is readily determined by one of skill in the art based upon the type of vector and the type of cell, and teachings widely available in the art. Infective agents may be introduced by a variety of means readily available in the art, including, e.g., nasal inhalation.

Methods of inhibiting gene expression using self-protected oligonucleotides of the invention may be combined with other knockdown and knockout methods, e.g., gene targeting, antisense RNA, ribozymes, double-stranded RNA (e.g., shRNA and siRNA) to further reduce expression of a target gene.

In different embodiments, target cells of the invention are primary cells, cell lines, immortalized cells, or transformed cells. A target cell may be a somatic cell or a germ cell. The target cell may be a non-dividing cell, such as a neuron, or it may be capable of proliferating in vitro in suitable cell culture conditions. Target cells may be normal cells, or they may be diseased cells, including those containing a known genetic mutation. Eukaryotic target cells of the invention include mammalian cells, such as, for example, a human cell, a murine cell, a rodent cell, and a primate cell. In one embodiment, a target cell of the invention is a stem cell, which includes, for example, an embryonic stem cell, such as a murine embryonic stem cell.

The self-protected polynucleotides and methods of the present invention may be used to treat any of a wide variety of diseases or disorders, including, but not limited to, inflammatory diseases, cardiovascular diseases, nervous system diseases, tumors, demyelinating diseases, digestive system diseases, endocrine system diseases, reproductive system diseases, hemic and lymphatic diseases, immunological diseases, mental disorders, muscoloskeletal diseases, neurological diseases, neuromuscular diseases, metabolic diseases, sexually transmitted diseases, skin and connective tissue diseases, urological diseases, and infections.

In certain embodiments, the methods are practiced on an animal, in particular embodiments, a mammal, and in certain embodiments, a human.

Accordingly, in one embodiment, the present invention includes methods of using a self-protected oligonucleotide for the treatment or prevention of a disease associated with gene deregulation, overexpression, or mutation. For example, a self-protected polynucleotide may be introduced into a cancerous cell or tumor and thereby inhibit expression of a gene required for or associated with maintenance of the carcinogenic/tumorigenic phenotype. To prevent a disease or other pathology, a target gene may be selected that is, e.g., required for initiation or maintenance of a disease/pathology. Treatment may include amelioration of any symptom associated with the disease or clinical indication associated with the pathology.

In addition, self-protected polynucleotides of the present invention are used to treat diseases or disorders associated with gene mutation. In one embodiment, a self-protected polynucleotide is used to modulate expression of a mutated gene or allele. In such embodiments, the mutated gene is the target of the self-protected polynucleotide, which will comprise a region complementary to a region of the mutated gene. This region may include the mutation, but it is not required, as another region of the gene may also be targeted, resulting in decreased expression of the mutant gene or mRNA. In certain embodiments, this region comprises the mutation, and, in related embodiments, the resulting self-protected oligonucleotides specifically inhibits expression of the mutant mRNA or gene but not the wild type mRNA or gene. Such a self-protected polynucleotide is particularly useful in situations, e.g., where one allele is mutated but another is not. However, in other embodiments, this sequence would not necessarily comprise the mutation and may, therefore, comprise only wild-type sequence. Such a self-protected polynucleotide is particularly useful in situations, e.g., where all alleles are mutated. A variety of diseases and disorders are known in the art to be associated with or caused by gene mutation, and the invention encompasses the treatment of any such disease or disorder with a self-protected polynucleotide. For example, in one embodiment, cystic fibrosis is treated using a self-protected polynucleotide that targets the cystic fibrosis transmembrane conductance regulator (CFTR) gene. In another embodiment, cancer is treated using a self-protected polynucleotide that targets a p53 gene or allele. In certain embodiment, the p53 gene or allele is a mutant p53 gene or allele.

In certain embodiments, a gene of a pathogen is targeted for inhibition. For example, the gene could cause immunosuppression of the host directly or be essential for replication of the pathogen, transmission of the pathogen, or maintenance of the infection. In addition, the target gene may be a pathogen gene or host gene responsible for entry of a pathogen into its host, drug metabolism by the pathogen or host, replication or integration of the pathogen's genome, establishment or spread of an infection in the host, or assembly of the next generation of pathogen. Methods of prophylaxis (i.e., prevention or decreased risk of infection), as well as reduction in the frequency or severity of symptoms associated with infection, are included in the present invention. For example, cells at risk for infection by a pathogen or already infected cells, particularly human immunodeficiency virus (HIV) infections, may be targeted for treatment by introduction of a self-protected polynucleotide according to the invention.

In other specific embodiments, the present invention is used for the treatment or development of treatments for cancers of any type. Examples of tumors that can be treated using the methods described herein include, but are not limited to, neuroblastomas, myelomas, prostate cancers, small cell lung cancer, colon cancer, ovarian cancer, non-small cell lung cancer, brain tumors, breast cancer, leukemias, lymphomas, and others.

The self-protected polynucleotides and expression vectors (including viral vectors and viruses) may be introduced into cells in vitro or ex vivo and then subsequently placed into an animal to affect therapy, or they may be directly introduced to a patient by in vivo administration. Thus, the invention provides methods of gene therapy, in certain embodiments. Compositions of the invention may be administered to a patient in any of a number of ways, including parenteral, intravenous, systemic, local, oral, intratumoral, intramuscular, subcutaneous, intraperitoneal, inhalation, or any such method of delivery. In one embodiment, the compositions are administered parenterally, i.e., intraarticularly, intravenously, intraperitoneally, subcutaneously, or intramuscularly. In a specific embodiment, the liposomal compositions are administered by intravenous infusion or intraperitoneally by a bolus injection.

Compositions of the invention may be formulated as pharmaceutical compositions suitable for delivery to a subject. The pharmaceutical compositions of the invention will often further comprise one or more buffers (e.g., neutral buffered saline or phosphate buffered saline), carbohydrates (e.g., glucose, mannose, sucrose, dextrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate.

The amount of self-protected oligonucleotides administered to a patient can be readily determined by a physician based upon a variety of factors, including, e.g., the disease and the level of self-protected oligonucleotides expressed from the vector being used (in cases where a vector is administered). The amount administered per dose is typically selected to be above the minimal therapeutic dose but below a toxic dose. The choice of amount per dose will depend on a number of factors, such as the medical history of the patient, the use of other therapies, and the nature of the disease. In addition, the amount administered may be adjusted throughout treatment, depending on the patient's response to treatment and the presence or severity of any treatment-associated side effects.

The invention further includes a method of identifying gene function in an organism comprising the use of a self-protected polynucleotide to inhibit the activity of a target gene of previously unknown function. Instead of the time consuming and laborious isolation of mutants by traditional genetic screening, functional genomics envisions determining the function of uncharacterized genes by employing the invention to reduce the amount and/or alter the timing of target gene activity. The invention may be used in determining potential targets for pharmaceutics, understanding normal and pathological events associated with development, determining signaling pathways responsible for postnatal development/aging, and the like. The increasing speed of acquiring nucleotide sequence information from genomic and expressed gene sources, including total sequences for the yeast, D. melanogaster, and C. elegans genomes, can be coupled with the invention to determine gene function in an organism (e.g., nematode). The preference of different organisms to use particular codons, searching sequence databases for related gene products, correlating the linkage map of genetic traits with the physical map from which the nucleotide sequences are derived, and artificial intelligence methods may be used to define putative open reading frames from the nucleotide sequences acquired in such sequencing projects.

In one embodiment, a self-protected oligonucleotide is used to inhibit gene expression based upon a partial sequence available from an expressed sequence tag (EST), e.g., in order to determine the gene's function or biological activity. Functional alterations in growth, development, metabolism, disease resistance, or other biological processes would be indicative of the normal role of the EST's gene product.

The ease with which a self-protected polynucleotide can be introduced into an intact cell/organism containing the target gene allows the present invention to be used in high throughput screening (HTS). For example, solutions containing self-protected polynucleotide that are capable of inhibiting different expressed genes can be placed into individual wells positioned on a microtiter plate as an ordered array, and intact cells/organisms in each well can be assayed for any changes or modifications in behavior or development due to inhibition of target gene activity. The function of the target gene can be assayed from the effects it has on the cell/organism when gene activity is inhibited. In one embodiment, self-protected polynucleotides of the invention are used for chemocogenomic screening, i.e., testing compounds for their ability to reverse a disease modeled by the reduction of gene expression using a self-protected polynucleotide of the invention.

If a characteristic of an organism is determined to be genetically linked to a polymorphism through RFLP or QTL analysis, the present invention can be used to gain insight regarding whether that genetic polymorphism might be directly responsible for the characteristic. For example, a fragment defining the genetic polymorphism or sequences in the vicinity of such a genetic polymorphism can be amplified to produce an RNA, a self-protected polynucleotide can be introduced to the organism, and whether an alteration in the characteristic is correlated with inhibition can be determined.

The present invention is also useful in allowing the inhibition of essential genes. Such genes may be required for cell or organism viability at only particular stages of development or cellular compartments. The functional equivalent of conditional mutations may be produced by inhibiting activity of the target gene when or where it is not required for viability. The invention allows addition of a self-protected polynucleotide at specific times of development and locations in the organism without introducing permanent mutations into the target genome. Similarly, the invention contemplates the use of inducible or conditional vectors that express a self-protected polynucleotide only when desired.

The present invention also relates to a method of validating whether a gene product is a target for drug discovery or development. A self-protected polynucleotide that targets the mRNA that corresponds to the gene for degradation is introduced into a cell or organism. The cell or organism is maintained under conditions in which degradation of the mRNA occurs, resulting in decreased expression of the gene. Whether decreased expression of the gene has an effect on the cell or organism is determined. If decreased expression of the gene has an effect, then the gene product is a target for drug discovery or development.

### C. Methods of Designing and Producing Self-Protected Polynucleotides

The self-protected polynucleotides of the present invention comprise a novel and unique set of functional sequences, arranged in a manner so as to adopt a secondary structure containing one or more double-stranded regions (typically a stem-loop structure), which imparts the advantages of the self-protected polynucleotides. Accordingly, in certain embodiments, the present invention includes methods of designing self-protected polynucleotide of the present invention. Such methods typically involve appropriate selection of the various sequence components of the self-protected polynucleotide.

In one embodiment, the basic design of self-protected polynucleotides is as follows:

DESIGN MOTIF:

(stemA)(loopA)(stemB)(X)(target)(X)(stemC)(loopB)(stemD)

x=optional spacer

Accordingly, in a related embodiment, a self-protected polynucleotide is designed as follows:

a. Start with target nucleotide sequence. The length and composition dictates the length and sequence composition of all stem and loop regions.

b. Stem A & D may need specific nucleotides for enzyme compatibility.

c. Build candidate Stem A & B with (4-12) nucleotides that have melting temperature dominant to equal length region of target. Stem strands have A-T, G-C complimentarity to each other.

d. Build candidate Stem C & D with (4-12) nucleotides that have melting temperature dominant to equal length region of target. Stem strands have A-T, G-C complimentarity to each other, but no complimentarity to Stem A & B.

e. Build loop candidates with (4-8) A-T rich nucleotides into loop A & B.

f. Form a contiguous sequence for each motif candidate.

g. Fold candidate sequence using software with desired parameters.

h. From output, locate structures with single stranded target regions which are flanked at either one or both ends with a desired stem/loop structure.

In one embodiment, a method of designing a polynucleotide sequence comprising one or more self-complementary regions for the regulation of expression of a target gene (i.e., a self-protected polynucleotide), includes: (a) selecting a first sequence 17 to 30 nucleotides in length and complementary to a target gene; and (b) selecting one or more additional sequences 12 to 48 nucleotides in length, which comprises self-complementary regions and which are non-complementary to the first sequence. In another embodiment, the method further includes (c) selecting one or more further additional sequences 2 to 12 nucleotides in length, which are non-complementary or semi-complementary to the target gene and which are non-complementary to the additional sequences selected in step (b).

These methods, in certain embodiments, include determining or predicting the secondary structure adopted by the sequences selected in step (b), e.g., in order to determine that they are capable of adopting a stem-loop structure.

Similarly, these methods can include a verification step, which comprises testing the designed polynucleotide sequence for its ability to inhibit expression of a target gene, e.g., in an in vivo or in vitro test system.

The invention further contemplates the use of a computer program to select sequences of a self-protected polynucleotide, based upon the complementarity characteristics described herein. The invention, thus, provides computer software programs, and computer readable media comprising said software programs, to be used to select self-protected polynucleotide sequences, as well as computers containing one of the programs of the present invention.

In certain embodiments, a user provides a computer with information regarding the sequence, location or name of a target gene. The computer uses this input in a program of the present invention to identify one or more appropriate regions of the target gene to target, and outputs or provides complementary sequences to use in the self-protected polynucleotide of the invention. The computer program then uses this sequence information to select sequences of the one or more self-complementary regions of the self-protected polynucleotide. Typically, the program will select a sequence that is not complementary to a genomic sequence, including the target gene, or the region of the self-protected polynucleotide that is complementary to the target mRNA. Furthermore, the program will select sequences of self-complementary regions that are not complementary to each other. When desired, the program also provides sequences of gap regions. Upon selection of appropriate sequences, the computer program outputs or provides this information to the user.

The programs of the present invention may further use input regarding the genomic sequence of the organism containing the target gene, e.g., public or private databases, as well as additional programs that predict secondary structure and/or hybridization characteristics of particular sequences, in order to ensure that the self-protected polynucleotide adopts the correct secondary structure and does not hybridize to non-target genes.

The present invention is based, in part, upon the surprising discovery that self-protected polynucleotides, as described herein, are extremely effective in reducing target gene expression. The self-protected polynucleotides offer significant advantages over previously described antisense RNAs, including increased stability or resistance to nucleases, and increased effectiveness. Furthermore, the self-protected polynucleotides of the invention offer additional advantages over traditional dsRNA molecules used for siRNA, since the use of self-protected polynucleotides substantially eliminates the off-target suppression associated with dsRNA molecules.

The practice of the present invention will employ a variety of conventional techniques of cell biology, molecular biology, microbiology, and recombinant DNA, which are within the skill of the art. Such techniques are fully described in the literature. See, for example, Molecular Cloning: A Laboratory Manual, 2nd Ed., ed. by Sambrook, Fritsch, and Maniatis (Cold Spring Harbor Laboratory Press, 1989); and DNA Cloning, Volumes I and II (D. N. Glover ed. 1985).

### EXAMPLE 1

### Design And Production of GL2-Targeted Self-Protected Polynucleotide pSUPER Expression Vector

Expression vectors designated GL2-1289 pSUPER, GL2-0209 pSUPER, and GL2-1532 pSUPER, which express self-protected polynucleotides targeting GL2 mRNA, were prepared using the pSUPER vector backbone according to the following design parameters. “5′-3′ non-coding” refers to the non-coding DNA strand in the plasmid; “5′-3′ RNA-trans” refers to the expressed RNA transcript from the plasmid; “5′-3′ RNA-struct” refers to the folded structure of the RNA transcript expressed from the plasmid; and “3′-5′ coding_as” refers to the coding DNA strand in the plasmid.

1. spDNA design logic for GL2-1289 pSUPER:

(SEQ ID NO:1)

5′-3′ non-coding = GATCCGCT CAAGA AGCGGATC
GTCAACTATGAAGAAGTGT AAACCTCAAT AGGTTTTTA

(SEQ ID NO:2)

5′-3′ RNA-trans. = GAUCCGCU CAAGA AGCGGAUC
GUCAACUAUGAAGAAGUGU AAACCUCAAU AGGUUU
5′-3′ Rna-struct = ((((((((.....))))))))
................... ((((((....)))))) (−16.90)

(SEQ ID NO:3)

3′-5′ coding_as = GGCGA GTTCT TCGCCTAG
CAGTTGATACTTCTTCACA TTTGGA GTTA TCCAAAAATTCGA

2. spDNA design logic for GL2-0209 pSUPER:

(SEQ ID NO:4)

5′-3′ non-coding = GATCCGCT CAAGA AGCGGATC
TGGGCTCAATACAAATCAC AAACCT CAAT AGGTTTTTA

(SEQ ID NO:5)

5′-3′ RNA-trans. = GAUCCGCU CAAGA AGCGGAUC
UGGGCUCAAUACAAAUCAC AAACCU CAAU AGGUUU
5′-3′ RNA-struct = ((((((((.....))))))))
................... ((((((....)))))) (−16.50)

(SEQ ID NO:6)

3′-5′ coding_as = GGCGA GTTCT TCGCCTAG
ACCCGAGTTATGTTTAGTG TTTGGA GTTATCCAAAAATTCGA

3. spDNA design logic for GL2-1532 pSUPER:

(SEQ ID NO:7)

5′-3′ non-coding = GATCCGCT CAAGA AGCGGATC
CACAACTCCTCCGCGCAAC AAACCT CAAT AGGTTTTTA

(SEQ ID NO:8)

5′-3′ RNA-trans. = GAUCCGCU CAAGA AGCGGAUC
CACAACUCCUCCGCGCAAC AAACCU CAAU AGGUUU
5′-3′ RNA-struct = ((((((((.....))))))))
................... ((((((....)))))) (−16.10)

(SEQ ID NO:9)

3′-5′ coding_as = GGCGA GTTCT TCGCCTAG
GTGTTGAGGAGGCGCGTTG TTTGGA GTTATCCAAAAATTCGA

The following oligonucleotide primers were used to PCR amplify regions of the GL2 gene for subcloning into the pSuper vector:

spDNA19_GL2-1289:

(SEQ ID NO:10)

GATCCGCTCAAGAAGCGGATCGTCAACTATGAAGAAGTGTAAACCTCAAT
AGGTTTTTA
spDNA19_GL2-1289_As:

(SEQ ID NO:11)

AGCTTAAAAACCTATTGAGGTTTACACTTCTTCATAGTTGACGATCCGCT
TCTTGAGCGG
spDNA19_GL2-0209:

(SEQ ID NO:12)

GATCCGCTCAAGAAGCGGATCTGGGCTCAATACAAATCACAAACCTCAAT
AGGTTTTTA
spDNA19_GL2-0209_As:

(SEQ ID NO:13)

AGCTTAAAAACCTATTGAGGTTTGTGATTTGTATTGAGCCCAGATCCGCT
TCTTGAGCGG
spDNA19_GL2-1532:

(SEQ ID NO:14)

GATCCGCTCAAGAAGCGGATCCACAACTCCTCCGCGCAACAAACCTCAAT
AGGTTTTTA
spDNA19_GL2-1532_As:

(SEQ ID NO:15)

AGCTTAAAAACCTATTGAGGTTTGTTGCGCGGAGGAGTTGTGGATCCGCT
TCTTGAGCGG.

The amplified sequences were subcloned into the pSuper vector (described generally in PCT Publication No. WO 01/36646; available from Oligoengine, Seattle, Wash.) using routine molecular and cell biology techniques.

### EXAMPLE 2

### Inhibition of Gene Expression by Self-Protected Polynucleotides

The inhibitory effects of the self-protected oligonucleotides of the present invention were demonstrated by examining their effect on in vivo gene expression using a human embryonic kidney cell line, 293-Lux, which stably produces luciferase (GL2 version). Cells were plated in 24 well dishes if 500 microliters of serum-containing medium. When the cells were approximately 60% confluent, they were transfected with 50 picomoles of a self-protected oligonucleotide comprising a region of RNA sequence complementary to the luciferase gene (or a negative control comprising a scrambled luciferase sequence) using 1 microliter of Mirus' TransIT-siQuest reagent. Specifically, the self-protected polynucleotides used included sp-19-1289, sp-19-209, sp-19-1532, sp-17-1289, and sp-17-209. For each of these self-protected polynucleotides, the first number (17 or 19) indicates the length of the region complementary to the target luciferase mRNA, and the second number (1289, 209, or 1532) indicates refers to the first base of the luciferase gene targeted in the complementary region. Cells were harvested at 24 hours, and lysates were read on a luminometer.

Of the five self-protected oligonucleotides tested, two reduced luciferase expression to between 50% and 60% of the control value, and two others reduced luciferase expression to approximately 75% or 80% of the control value (Table 1).

TABLE 1
Self-protected polynucleotide inhibition of luciferase expression

| Sample | Raw RLUs | % Expression |
|---|---|---|
| Scramble Control | 15336976 | 100.00% |
| sp-19-1289 | 16165063 | 105.40% |
| sp-19-209 | 8039347 | 52.42% |
| sp-19-1532 | 8900588 | 58.03% |
| sp-17-1289 | 11506895 | 75.03% |
| sp-17-209 | 12553289 | 81.85% |

These data demonstrate that the self-protected polynucleotide sequences of the present invention are efficient in reducing in vivo gene expression, establishing that they can be used for a variety of purposes, including the treatment of diseases and disorders associated with gene overexpression.

### EXAMPLE 3

### Inhibition of P53 Gene Expression in Mammalian Cells Using Transit-TKO siRNA Transfection Reagent

The effectiveness of the self-protected oligonucleotides of the present invention in reducing p53 gene expression in mammalian cells was demonstrated using cultured cells. 75% confluent 293-H cells were transfected with 2 microliters of TransIT-TKO siRNA Transfection Reagent (Mirus Bio Corporation, Madison, Wis.) and 50 nmoles of an spRNA oligonucleotide listed in Table 2, or a p53 siRNA oligonucleotide, in a 24 well plate containing 300 ul of DMEM/10% BSA.

The p53 gene sequence (Genbank Accession No. AB082923) contains a previously published RNAi target sequence within its transcribed mRNA. The mRNA sequence of this target site is GACUCCAGUGGUAAUCUAC (SEQ ID NO:16). siRNA and spRNA oligonucleotides designated “siRNA p53_public” and spRNA p53_public(9.0)” targeting this sequence were prepared. These oligonucleotides contained the following sequences, and spRNA p53-public(9.0) possessed the indicated structure:

sIRNA p53_public:
sense 5-3′:

(SEQ ID NO:17)

GACUCCAGUGGUAAUCUACTT
as 5-3′:

(SEQ ID NO:18)

GUAGAUUACCACUGGAGUCTT
spRNA p53_public(9.0) 5′-3′:

(SEQ ID NO:19)

CCCUUAUAGAGGGGUAGAUUACCACUGGAGUCGCGUUAUAGACGC
structure:
((((((...))))))...((((.((...))))))(((((...)))))

Additional spRNA oligonucleotides were prepared to target the following p53 mRNA site, which was determined to be non-folding (n..19): UGCCCUCAACAAGAUGUUU (SEQ ID NO:20).

spRNA p53_{—}83 contains a short stem/loop structure on both sides of the n..19 binding motif and includes the following 5′-3′ sequence and structure:

(SEQ ID NO:21)

UCCGAGUUAGACUCGGAAAACAUCUUGUUGAGGGCAGGAACCUUAUAUGG
UUCC
structure:
((((((.....))))))...................
((((((......))))))

spRNA p53_{—}83 NO-5′ contains short stem/loop structure on the 3′ side of the n..19 binding motif and includes the following 5′-3′ sequence and structure:

(SEQ ID NO:22)

AAACAUCUUGUUGAGGGCAGGAACCUUAUAUGGUUCC
structure: ...................((((((......))))))

spRNA p53_{—}83_Long contains longer stem/loop structures on both sides of the n..19 binding motif and includes the following 5′-3′ sequence and structure:

(SEQ ID NO:23)

UCCAGGAGUUAGACUCCUGGAAAACAUCUUGUUGAGGGCAGGAGCACCUU
AUAUGGUGCUCC
structure:
((((((((.....))))))))...................
((((((((......))))))))

spRNA p53_{—}83_Long_NO-5′ contains a longer stem/loop structure on the 3′ side of the n..19 binding motif and includes the following 5′-3′ sequence and structure:

(SEQ ID NO:24)

AAACAUCUUGUUGAGGGCAGGAGCACCUUAUAUGGUGCUCC
structure:
...................((((((((......))))))))

Cells were harvested at 48 hours in 100 microliters of passive lysis buffer. 100 microliters of a 1:40 dilution was tested using an Assay Designs p53 TiterZyme EIA kit (Ann Arbor, Mich.) to determine the amount of p53 present. The results shown in Table 2 are provided in picograms of p53 as estimated from the control standards used to prepare a standard curve at the time of the assay. The percentage of p53 Knockdown is based on picograms of p53 as compared to a mock transfected well.

TABLE 2
Self-protected polynucleotide inhibition of p53 expression

| Sample | Pg p53 | % Knockdown |
|---|---|---|
| Mock Transfection | 235 | 0 |
| Scramble Control | 230 | 2 |
| spRNA p53_public(−9.0) | 130 | 45 |
| spRNA p53-83 NO-5′ | 150 | 36 |
| spRNA p53-83 | 160 | 32 |
| spRNA p53_83_Long_NO-5′ | 150 | 36 |
| spRNA p53_83_Long | 80 | 66 |

In comparison, the results obtained using siRNA p53 public were 160 pg of p53 (32% Knock down of endogenous p53).

These results demonstrate that the self-protected oligonucleotides of the present invention are useful in reducing target gene expression in mammalian cells, and support their use in treating diseases caused by gene overexpression, deregulation, inappropriate expression, or gene mutation, including, but not limited to, tumors caused by p53 mutation. In addition, they indicate that self-protected polynucleotides of the present invention provide increased efficacy as compared to siRNA oligonucleotides in reducing target gene expression.

### EXAMPLE 4

### Inhibition of P53 Gene Expression in Mammalian Cells Using SIQuest Reagent

The effectiveness of the self-protected oligonucleotides in reducing p53 gene expression in mammalian cells was demonstrated using cultured cells using a different transfection reagent. 65% confluent 293-H cells were transfected with 1 microliter of TransIT®-siQUEST™ reagent (Mirus Bio Corporation, Madison, Wis.) and 50 nmoles of an spRNA indicated in Table 3, or a p53 siRNA, in a 24 well plate containing 300 ul of DMEM/10% BSA.

Cells were harvested at 48 hours in 100 microliters of passive lysis buffer. 100 microliters of a 1:40 dilution was tested using an Assay Designs p53 TiterZyme EIA kit to determine the amount of p53 present. The results shown in Table 3 indicate picograms of p53 as estimated from the control standards used to prepare a standard curve at the time of the assay. The percentage of p53 Knockdown is based on picograms of p53 as compared to a mock transfected well.

TABLE 3
Self-protected polynucleotide inhibition of p53 expression

| Sample | Pg p53 | % Knockdown |
|---|---|---|
| Mock Transfection | 335 | 0 |
| Scramble Control | 305 | 6 |
| spRNA p53-83 NO-5′ | 315 | 3 |
| spRNA p53-83 | 345 | −3 |
| spRNA p53_83_Long_NO-5′ | 255 | 22 |
| spRNA p53_83_Long | 235 | 28 |

In comparison, the results obtained using siRNA p53 public were 215 pg p53 (34% knock down of endogenous p53).

These results demonstrate that the self-protected oligonucleotides of the present invention are useful in reducing target gene expression in mammalian cells, and support their use in treating diseases caused by gene overexpression, deregulation, inappropriate expression, or gene mutation, including, but not limited to, tumors caused by p53 mutation. In addition, they indicate that self-protected polynucleotides of the present invention provide increased efficacy as compared to siRNA oligonucleotides in reducing target gene expression.

All of the above U.S. patents, U.S. patent application publications, U.S. patent applications, foreign patents, foreign patent applications and non-patent publications referred to in this specification and/or listed in the Application Data Sheet are incorporated herein by reference, in their entirety.

From the foregoing it will be appreciated that, although specific embodiments of the invention have been described herein for purposes of illustration, various modifications may be made without deviating from the spirit and scope of the invention.

1. An isolated polynucleotide comprising a region having a sequence complementary to a target gene or mRNA sequence and one or more self-complementary regions.

2. The polynucleotide of claim 1, wherein said polynucleotide comprises two or more self-complementary regions.

3. The polynucleotide of claim 1, wherein said polynucleotide comprises RNA.

4. The polynucleotide of claim 1, wherein said polynucleotide comprises DNA.

5. The polynucleotide of claim 1, wherein said polynucleotide comprises a peptide nucleic acid.

6. The polynucleotide of claim 1, wherein said self-complementary regions are located at the 5′ or 3′ or both ends of the polynucleotide.

7. The polynucleotide of claim 1, further comprising one or more additional regions of sequence complementary to a target gene or mRNA sequence, wherein said regions of sequence complementary to a target gene or mRNA sequence are separated by self-complementary regions are located at the 5′ or 3′ or both ends of the polynucleotide.

8. The polynucleotide of claim 7, wherein said regions of sequence complementary to a target gene or mRNA sequence are complementary to the same target gene or mRNA sequence.

9. The polynucleotide of claim 7, wherein said regions of sequence complementary to a target gene or mRNA sequence are complementary to two or more different genes or mRNA sequences.

10. The polynucleotide of claim 1, further comprising a second sequence that is non-complementary or semi-complementary to a target gene or mRNA sequence and non-complementary to a self-complementary region, wherein said second sequence is located between the self-complementary region and the sequence complementary to a target gene or mRNA sequence.

11. The polynucleotide of claim 1, wherein said self-complementary region comprises a stem-loop structure.

12. The polynucleotide of claim 1, wherein said self-complementary region does not complement the sequence complementary to a target gene or mRNA sequence.

13. The polynucleotide of claim 1, wherein said polynucleotide comprises two self-complementary regions, and wherein said two self-complementary regions do not complement each other.

14. The polynucleotide of claim 1, wherein said sequence complementary to a target gene or mRNA sequence comprises at least 17 nucleotides.

15. The polynucleotide of claim 14, wherein said sequence complementary to a target gene or mRNA sequence comprises 17 to 30 nucleotides.

16. The polynucleotide of claim 14, wherein said self-complementary region comprises at least 5 nucleotides.

17. The polynucleotide of claim 14, wherein said self-complementary region comprises at least 24 nucleotides.

18. The polynucleotide of claim 14, wherein said self-complementary region comprises 12 to 48 nucleotides.

19. The polynucleotide of claim 11, wherein said loop comprises at least 4 nucleotides.

20. An array comprising a plurality of polynucleotides of claim 1.

21. An expression vector encoding a polynucleotide of claim 1.

22. A composition comprising a physiologically acceptable carrier and a polynucleotide of claim 1.

23. A method for reducing the expression of a gene, comprising introducing an isolated polynucleotide of claim 1 into a cell.

24. The method of claim 23, wherein the cell is plant, animal, protozoan, viral, bacterial, or fungal.

25. The method of claim 23, wherein the cell is mammalian.

26. The method of claim 23, wherein the isolated polynucleotide is introduced directly into the cell.

27. The method of claim 23, wherein the isolated polynucleotide is introduced extracellularly by a means sufficient to deliver the isolated polynucleotide into the cell.

28. A method for treating a disease, comprising introducing an isolated polynucleotide of claim 1 into a cell, wherein expression of the gene or mRNA is associated with the disease.

29. The method of claim 28, wherein the disease is a cancer.

30. A method of treating an infection in a patient, comprising introducing into the patient the isolated polynucleotide of claim 1, wherein the isolated polynucleotide mediates entry, replication, integration, transmission, or maintenance of an infective agent.

31. A method for identifying a function of a gene, comprising:
(a) introducing into a cell the isolated polynucleotide of claim 1, wherein the isolated polynucleotide inhibits expression of the gene; and
(b) determining the effect of step (a) on a characteristic of the cell, thereby determining the function of the gene.

32. The method of claim 31, wherein the method is performed using high throughput screening.

33. A method of designing a polynucleotide sequence comprising one or more self-complementary regions for the regulation of expression of a target gene or mRNA, comprising:
(a) selecting a first sequence 17 to 30 nucleotides in length and complementary to a target gene or mRNA;
(b) selecting one or more additional sequences 12 to 48 nucleotides in length, which comprises self-complementary regions and which are non-complementary to the first sequence; and
(c) selecting one or more further additional sequences 2 to 12 nucleotides in length, which are non-complementary or self-complementary to the target gene or mRNA and which are non-complementary to the additional sequences selected in step (b),
thereby designing a polynucleotide sequence for the regulation of expression of a target gene or mRNA.

34. The polynucleotide of claim 1, wherein said polynucleotide exhibits an increased half-life in vivo, as compared to the same polynucleotide lacking the one or more self-complementary regions.

35. A method for treating a disease, comprising introducing an isolated polynucleotide of claim 1 into a cell, wherein said gene or mRNA comprises one or more mutations as compared to a corresponding wild-type gene or mRNA.

36. The method of claim 35, wherein said disease is cystic fibrosis.

37. A method of modulating the expression of a mutated gene or mRNA in a cell, comprising introducing a polynucleotide of claim 1 into a cell, wherein said target gene or mRNA sequence comprises a region of said mutated gene or mRNA.

38. The method of claim 37, wherein said mutated gene or mRNA is associated with cystic fibrosis.

39. The method of claim 38, wherein said mutated mRNA is an mRNA expressed from a gene encoding a mutant Cystic Fibrosis Transmembrane Conductance Regulator (CFTR) polypeptide.

40. The method of claim 37, wherein said mutated gene or mRNA is associated with a tumor.

41. The method of claim 40, wherein said mutated mRNA is an mRNA expressed from a gene encoding a mutant p53 polypeptide.

42. The method of claim 23, wherein said target gene is a transactivator that drives the expression of a second gene.

43. The method of claim 23, wherein said target gene is a repressor that inhibits expression of a second gene.


SEQUENCE LISTING (24 sequences)

SEQ ID NO: 1 - DNA, 59 bp, Artificial Sequence
  Sequence from GL2-1289 pSUPER
gatccgctcaagaagcggatcgtcaactatgaagaagtgtaaacctcaataggttttta

SEQ ID NO: 2 - RNA, 56 nt, Artificial Sequence
  Expressed RNA transcript from GL2-1289 pSUPER
gauccgcucaagaagcggaucgucaacuaugaagaaguguaaaccucaauagguuu

SEQ ID NO: 3 - DNA, 60 bp, Artificial Sequence
  Coding strand from GL2-1289 pSUPER
ggcgagttcttcgcctagcagttgatacttcttcacatttggagttatccaaaaattcga

SEQ ID NO: 4 - DNA, 59 bp, Artificial Sequence
  non-coding strand from GL2-0209 pSUPER
gatccgctcaagaagcggatctgggctcaatacaaatcacaaacctcaataggttttta

SEQ ID NO: 5 - RNA, 56 nt, Artificial Sequence
  RNA transcript expressed from GL2-0209 pSUPER
gauccgcucaagaagcggaucugggcucaauacaaaucacaaaccucaauagguuu

SEQ ID NO: 6 - DNA, 60 bp, Artificial Sequence
  Coding sequence from GL2-0209 pSUPER
ggcgagttcttcgcctagacccgagttatgtttagtgtttggagttatccaaaaattcga

SEQ ID NO: 7 - DNA, 59 bp, Artificial Sequence
  Non-Coding sequence from GL2-1532 pSUPER
gatccgctcaagaagcggatccacaactcctccgcgcaacaaacctcaataggttttta

SEQ ID NO: 8 - RNA, 56 nt, Artificial Sequence
  RNA transcript expressed from GL2-1532 pSUPER
gauccgcucaagaagcggauccacaacuccuccgcgcaacaaaccucaauagguuu

SEQ ID NO: 9 - DNA, 60 bp, Artificial Sequence
  Coding sequence from GL2-1532 pSUPER
ggcgagttcttcgcctaggtgttgaggaggcgcgttgtttggagttatccaaaaattcga

SEQ ID NO: 10 - DNA, 59 bp, Artificial Sequence; identical to SEQ ID NO: 1
  Oligonucleotide primer for PCR amplification of GL2 regions

SEQ ID NO: 11 - DNA, 60 bp, Artificial Sequence
  Oligonucleotide primer for PCR amplification of GL2 regions
agcttaaaaacctattgaggtttacacttcttcatagttgacgatccgcttcttgagcgg

SEQ ID NO: 12 - DNA, 59 bp, Artificial Sequence; identical to SEQ ID NO: 4
  Oligonucleotide primer for PCR amplification of GL2 regions

SEQ ID NO: 13 - DNA, 60 bp, Artificial Sequence
  Oligonucleotide primer for PCR amplification of GL2 regions
agcttaaaaacctattgaggtttgtgatttgtattgagcccagatccgcttcttgagcgg

SEQ ID NO: 14 - DNA, 59 bp, Artificial Sequence; identical to SEQ ID NO: 7
  Oligonucleotide primer for PCR amplification of GL2 regions

SEQ ID NO: 15 - DNA, 60 bp, Artificial Sequence
  Oligonucleotide primer for PCR amplification of GL2 regions
agcttaaaaacctattgaggtttgttgcgcggaggagttgtggatccgcttcttgagcgg

SEQ ID NO: 16 - RNA, 19 nt, Homo sapiens
gacuccagugguaaucuac

SEQ ID NO: 17 - DNA, 21 bp, Artificial Sequence
  Oligonucleotide
gacuccagugguaaucuactt

SEQ ID NO: 18 - DNA, 21 bp, Artificial Sequence
  Oligonucleotide
guagauuaccacuggaguctt

SEQ ID NO: 19 - RNA, 45 nt, Artificial Sequence
  Oligonucleotide
cccuuauagagggguagauuaccacuggagucgcguuauagacgc

SEQ ID NO: 20 - RNA, 19 nt, Homo sapiens
ugcccucaacaagauguuu

SEQ ID NO: 21 - RNA, 54 nt, Artificial Sequence
  Oligonucleotide
uccgaguuagacucggaaaacaucuuguugagggcaggaaccuuauaugguucc

SEQ ID NO: 22 - RNA, 37 nt, Artificial Sequence
  Oligonucleotide
aaacaucuuguugagggcaggaaccuuauaugguucc

SEQ ID NO: 23 - RNA, 62 nt, Artificial Sequence
  Oligonucleotide
uccaggaguuagacuccuggaaaacaucuuguugagggcaggagcaccuuauauggugcucc

SEQ ID NO: 24 - RNA, 41 nt, Artificial Sequence
  Oligonucleotide
aaacaucuuguugagggcaggagcaccuuauauggugcucc